(19) United States
(12) Patent Application Publication
(10) Pub. No.: US 2024/0042079 A1
(43) Pub. Date: Feb. 8, 2024

(54) SHOE CARE APPARATUS

(71) Applicant: SAMSUNG ELECTRONICS CO., LTD., Suwon-si (KR)

(72) Inventors: Daegeon KIM, Suwon-si (KR); Donggpi; Seo, Suwon-si (KR); Jaeryong Park, Suwon-si (KR)

(73) Assignee: SAMSUNG ELECTRONICS CO., LTD., Suwon-si (KR)

(21) Appl. No.: 18/381,502

(22) Filed: Oct. 18, 2023

Related U.S. Application Data
(63) Continuation of application No. PCT/KR2022/008844, filed on Jun. 22, 2022.

(30) Foreign Application Priority Data
Jul. 7, 2021 (KR) 10-2021-0089016

(51) Int. Cl. A61L 2/10; A47L 23/20; A61L 2/26; A61L 2/14; A47B 61/04; F25B 30/02

(52) U.S. Cl. CPC A61L 2/10; A47L 23/205; A61L 2/26; A61L 2/14; A47B 61/04; F25B 30/02; A61L 2202/11; A61L 2202/14

(57) ABSTRACT

Disclosed is a shoe care apparatus including: a cabinet; a chamber provided in the cabinet for accommodating a shoe; a machine room provided to accommodate a heat pump which heats the air supplied to the chamber and dehumidifies the air discharged from the chamber; an air supply duct which connects the chamber to the machine room; an air supply port which is provided in the sidewall of the chamber such that the air flowing along the air supply duct is supplied into the chamber through the air supply port; a support member which is provided on the sidewall of the chamber to be adjacent to the air supply port; and a shoe hanger which includes a pair of support arms that are accommodated in the chamber, that are configured to be inserted into , is detachably installed on the support member, and has a hollow inside such that the air supplied from the air supply port may flow therein, wherein the shoe hanger has a sterilization device provided in the hollow inside and the support member is provided with a power supply part which applies power to the sterilization device.

### CROSS-REFERENCE TO RELATED APPLICATIONS

This application is a Bypass Continuation Application of PCT International Application No. PCT/KR2022/008844, filed on Jun. 22, 2022, which is based on and claims priority to Korean Patent Application No. 10-2021-0089016, filed on Jul. 7, 2021, in the Korean Intellectual Property Office, the disclosure of which are incorporated by reference herein in their entireties.

### BACKGROUND

### 1. Field

The present disclosure relates to a shoe care apparatus, and more specifically, to a shoe support device allowing even the inside of a shoe to be sterilized and a shoe care apparatus including the same.

### 2. Description of Related Art

In general, shoes are easily become dirty due to foreign substances entering the shoes through the opening or even due to sock, and also easily contaminated due to sweat inside the shoes when worn for walking or exercising, resulting in poor hygiene. Due to the structure of the shoes in which the inside is deeply formed, it is difficult to wash or sterilize the inside of the shoe.

A shoe care apparatus is an apparatus for caring for shoes, such as drying shoes, cleaning shoes, and removing odors from shoes.

Sterilization in the shoe care apparatus may be performed using an ultraviolet (UV) lamp, and the like, but since a sterilization device is located in an upper central portion of the inside of the shoe care apparatus, there is a limitation in that only the outside of the shoe, that is, the upper and outsole portions, are sterilized.

In addition, since the UV lamp in the upper central portion of the shoe care apparatus is located in a section having almost no air flow, and several pairs of shoes need to sterilized with a single UV lamp, the operation takes a long time, which causes the temperature of the UV lamp to rise, thereby shortening the lifespan of the UV lamp.

In addition, the overheating of the ultraviolet lamp may lead to damage to the shoes.

### SUMMARY

Provides is a shoe care apparatus capable of effectively sterilizing the inside and outside of shoes.

It is an aspect of the disclosure to provide a shoe support device that allows even the inside of a shoe to be sterilized, and a shoe care apparatus including the same.

It is an aspect of the disclosure to provide a shoe care apparatus that guides air to an ultraviolet lamp, thereby preventing shoes from being damaged due to overheating.

It is an aspect of the disclosure to provide a shoe care apparatus capable of utilizing a structure for applying power to an ultraviolet lamp as a mounting detection device for a shoe support device.

It is an aspect of the disclosure to provide a shoe care apparatus capable of identifying whether a shoe support device having a detachable structure is mounted at an accurate position.

In accordance with an aspect of the disclosure, there is provided a shoe care apparatus including: a cabinet; a chamber provided in the cabinet configured to accommodate shoes; a machine room configured to accommodate a heat pump for heating air supplied to the chamber and dehumidifying air discharged from the chamber; an air supply duct configured to connect the chamber and the machine room; an air supply port formed on a sidewall of the chamber such that air flowing along the air supply duct is supplied into the chamber through the air supply port; a support member, provided on the side wall of the chamber, disposed adjacent to the air supply port; and a shoe hanger including a pair of support arms accommodated in the chamber, that are configured to be inserted into shoes, the shoe hanger is detachably mounted to the support member and has a hollow formed therein to allow air supplied from the air supply port to flow therethrough, wherein the shoe hanger is provided with a sterilization device disposed inside the hollow, and the support member is provided with a power supply part for applying power to the sterilization device.

The shoe care apparatus may include a shoe hanger body including the pair of support arms configured to protrude to support the shoes; a passage formed inside the shoe hanger body while communicating with the air supply duct such that air moves to the pair of support arms; and a plurality of holes connected to the passage and formed on outer surfaces of the pair of support arms to supply air into the chamber.

The sterilization device may be disposed on the passage.

The sterilization device may include at least one of an ultraviolet lamp or an ion generator.

The UV lamp or the ion generator may be disposed inside the pair of support arms.

The pair of support arms may further include an installation bracket fixed to an inner upper surface of the pair of support arms such that the UV lamp or the ion generator is positioned on the passage.

The power supply part may be configured to supply the sterilization device with power, and may be disposed on at least one of inner walls of a lateral side, an upper side, or a lower side of the support member.

The shoe hanger may further include a power connection part that corresponds to the power supply part.

Based on the shoe hanger being in a first position P1 of being coupled to the support member, the power connection part and the power supply part may be in contact with each other, and the sterilization device may be turned on.

Based on the shoe hanger being in a second position P2 of being separated from the support member, the power connection part and the power supply part may be separated from each other, and the sterilization device may be turned off

The power supply part may further include a contact terminal disposed on the at least one of the inner walls of the lateral side, the upper side, or the lower side of the support member to be in contact with the power connection part.

The power supply part may further include an elastic body configured to elastically support the contact terminal.

The shoe care apparatus may further include: a heat exchanger configured to heat air introduced from the chamber including a compressor; and a control part configured to alternatively turn on the compressor and the sterilization device.

The control part may be configured to: based on the compressor being turned off, turn on the sterilization device; and based on the sterilization device being turned off, turn on the compressor.

In accordance with an aspect of the disclosure, there is provided a shoe care apparatus including: an output part including at least one of a display part or a speaker; and a control part configured to control the output part to output a visual notification or an audible notification notifying that the shoe hanger has been separated from the support member because power is not being applied to the sterilization device through the power supply part.

The control part may, in response to receiving a user input for starting a course of the shoe care apparatus, identify whether power is applied to the sterilization device through the power supply part.

In accordance with an aspect of the disclosure, there is provided a shoe care apparatus: including an output part including at least one of a display and a speaker; and a control part configured to control the output part based on a power supply state of the power supply part, wherein the power supply part is disposed on an upper inner wall of the support member, and the control part, because power is not being applied to the sterilization device through the power supply part, control the output part to output a visual notification or an audible notification notifying that shoes are not hung on the shoe hanger.

### Advantageous Effects

According to the concept of the present disclosure, due to a shoe support device that enables internal sterilization, the inside and outside of shoes can be sterilized, thereby providing more hygienic shoes.

In addition, since an UV lamp installed inside a shoe support device guiding air into shoes and allowing shoes to be hung thereon is implemented to always contact air, the temperature rise of the UV lamp can be suppressed, shoes can be prevented from being damaged due to overheating, and the issue of a shortened lamp lifespan can be addressed.

In addition, since a power supply structure for applying power to a UV lamp installed inside a shoe support device is used as a mounting detection device for of the shoe support device to thereby detect whether the shoe support device is correctly mounted can be detected, thereby allowing the shoe support device to be mounted in the correct position, by which the performance degradation issue that occurs when the mounting position is deviated can be addressed, and the optimum performance can be always secured.

### BRIEF DESCRIPTION OF DRAWINGS

The above and other aspects and features of certain embodiments of the present disclosure will be more apparent from the following description taken in conjunction with the accompanying drawings, in which:

FIG. 1 is a diagram illustrating a shoe care apparatus according to an embodiment of the present disclosure.

FIG. 2 is a perspective view illustrating a state in which a door of the shoe care apparatus shown in FIG. 1 is opened.

FIG. 3 is a cross-sectional view of the shoe care apparatus shown in FIG. 2, when viewed from the front.

FIG. 4 is a perspective view illustrating components related to a sterilization device of the shoe care apparatus shown in FIG. 3.

FIG. 5 is an exploded view illustrating a first sterilization portion of the shoe care apparatus shown in FIG. 4.

FIG. 6 is a perspective view illustrating a second sterilization portion mounted on a shoe hanger shown in FIG. 4.

FIG. 7 is an exploded perspective view illustrating a second sterilization portion mounted on the shoe hanger shown in FIG. 6.

FIG. 8 is a cross-sectional view illustrating a second sterilization portion mounted on the shoe hanger shown in FIG. 6.

FIG. 9 is a view illustrating a state in which the shoe hanger shown in FIG. 4 is being mounted on a support member.

FIG. 10 is a view illustrating the shoe hanger shown in FIG. 4 mounted on the support member, which shows a state in which a power connection part is being connected to a power supply part.

FIG. 11 is a cross-sectional view illustrating a coupled state of the power supply part and the power connection part shown in FIG. 10.

FIG. 12 is a diagram illustrating a power supply part and a power connection part mounted in an alternative position according to another embodiment of the present disclosure.

FIG. 13 is a diagram illustrating a power supply part and a power connection part mounted in an alternative position according to another embodiment of the present disclosure.

FIG. 14 is an exploded perspective view illustrating a shoe hanger with a second sterilization portion mounted thereon according to another embodiment of the present disclosure.

FIG. 15 is a cross-sectional view illustrating the second sterilization portion mounted on the shoe hanger shown in FIG. 14.

FIG. 16 is a control block diagram illustrating a shoe care apparatus according to an embodiment of the present disclosure.

FIG. 17 is a flowchart showing an example of course operations of a shoe care apparatus according to an embodiment of the present disclosure.

FIG. 18 is a flowchart illustrating an example of outputting a warning notification by a shoe care apparatus according to an embodiment of the present disclosure.

### DETAILED DESCRIPTION

Embodiments described in the specification and configurations shown in the accompanying drawings are merely examples of the disclosure, and various modifications may replace the embodiments and the drawings of the disclosure at the time of filing of the application.

Further, identical symbols or numbers in the drawings of the disclosure denote components or elements configured to perform substantially identical functions.

Further, terms used herein are only for the purpose of describing particular embodiments and are not intended to limit to the disclosure. The singular form is intended to include the plural form as well, unless the context clearly indicates otherwise. It should be further understood that the terms “include,” “including,” “have,” and/or “having” specify the presence of stated features, integers, steps, operations, elements, components, and/or groups thereof, but do not preclude the presence or addition of one or more other features, integers, steps, operations, elements, components, and/or groups thereof

Further, it should be understood that, although the terms “first,” “second,” etc. may be used herein to describe various elements, the elements are not limited by the terms, and the terms are only used to distinguish one element from another. For example, a first element could be termed a second element, and similarly, a second element could be termed a first element without departing from the scope of the disclosure. The term “and/or” includes combinations of one or all of a plurality of associated listed items.

Meanwhile, the terms “front”, “rear”, “left”, and “right” used in the following description are defined based on the drawings, and the shape and location of each component are not limited by these terms.

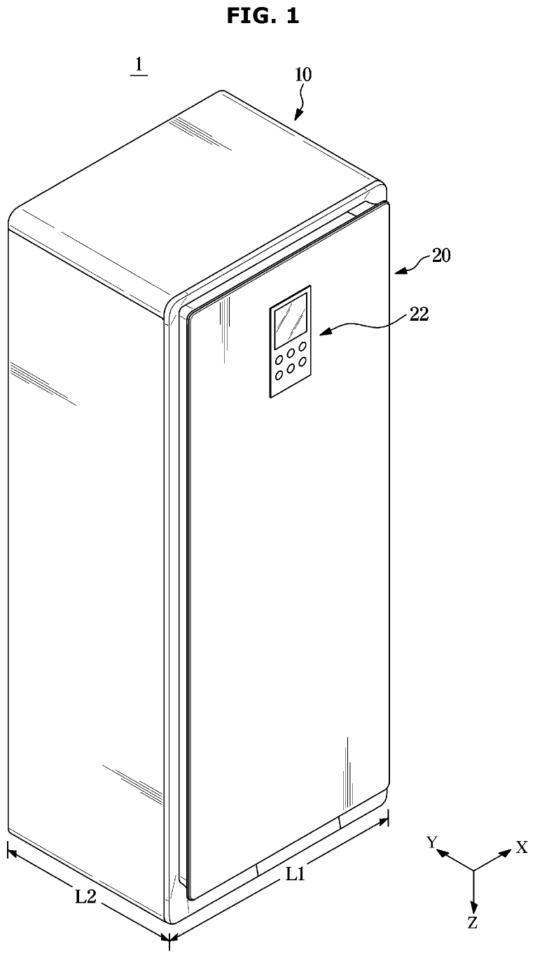

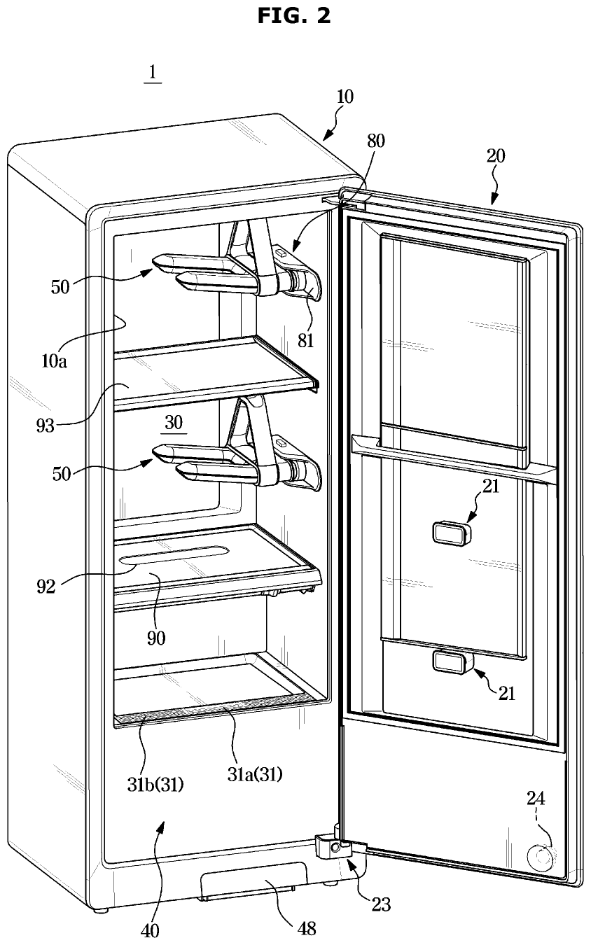

Specifically, as shown in FIGS. 1 and 2, when a door of a shoe care apparatus is opened, the front is a side from which inside of the shoe care apparatus is viewed.

Hereinafter, embodiments according to the present disclosure will be described in detail with reference to the accompanying drawings.

A shoe care apparatus may include various devices for dehumidifying a certain space, such as a clothes care device or a shoe care apparatus. However, for convenience of description, the following description will be made in relation to a shoe care apparatus.

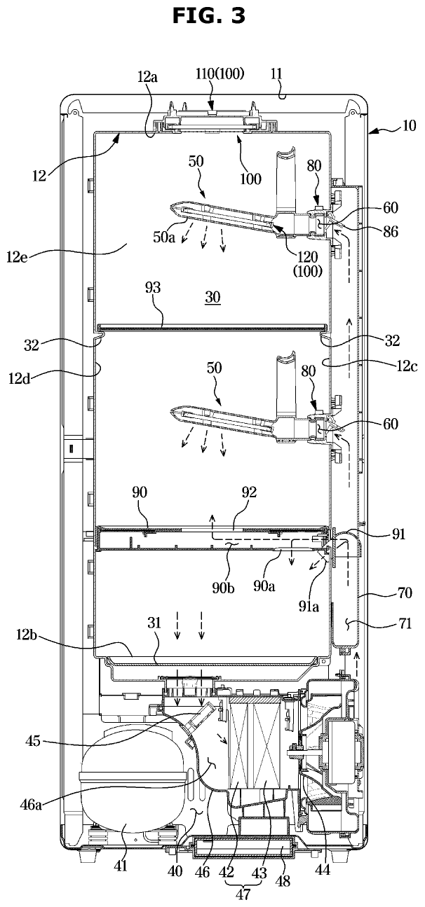

FIG. 1 is a diagram illustrating a shoe care apparatus according to an embodiment of the present disclosure. FIG. 2 is a perspective view illustrating a state in which a door of the shoe care apparatus shown in FIG. 1 is opened. FIG. 3 is a cross-sectional view of the shoe care apparatus shown in FIG. 2, when viewed from the front.

Referring to FIGS. 1 to 3, a shoe care apparatus 1 may include a main body 10 forming an external appearance and a door 20 rotatably coupled to the main body 10.

The main body 10 may be provided in a rectangular parallelepiped shape with an open front. An opening 10a may be formed on the open front of the main body 10. The door 20 may be rotatably coupled to the main body 10 to open and close the open front of the main body 10. The door 20 may be coupled to the main body 10 through a hinge 23 or the like.

The door 20 may include a hanging member 21 provided on a surface facing an inside of a shoe care room 30. The hanging member 21 may be provided in at least one unit of the shoe care room 30. The hanging member 21 may be formed to allow a handle of a shoe support device 50, which will be described below, to be hung such that the shoe support device 50 may be easily stored. However, the use of the hanging member 21 is not limited thereto, and the hanging member may be used for hanging other components.

The door 20 may further include a display 22 provided on the front surface of the shoe care apparatus 1. The user may set an appropriate care course for each shoes in the display 22 according to the type of the shoes inside the shoe care room 30. With such a configuration, the temperature and humidity of the shoe care room 30 may be adjusted and damage to shoes may be reduced.

The door 20 may be provided in a lower portion thereof with an opening/closing detection sensor 24 for detecting opening/closing of the door 20. The opening/closing detection sensor 24 may be a reed switch. However, it is not limited thereto, and the opening/closing detection sensor 24 may include various types of sensors.

The main body 10 may include an external cabinet 11 and an inner cabinet 12 disposed inside the external cabinet 11. The inner cabinet 12 may be referred to as a cabinet 12.

The main body 10 may be formed such that the length of the front surface extending in the first direction X and the length of the side surface extending in the second direction Y are different. That is, the length L1 of the front surface may be longer than the length L2 of the side surface. Accordingly, even for a narrow entrance, the shoe care apparatus 1 may be easily installed. The length of the front surface may be the first length L1, and the length of the side surface may be the second length L2.

The shoe care apparatus 1 may include a shoe hanger 50 provided inside the shoe care room 30 to support shoes. The shoe care room 30 may be referred to as a chamber 30. The chamber 30 may be provided to accommodate shoes.

The shoe care room 30 may form a space in which shoes are accommodated. The shoe care room 30 may be provided by the inner cabinet 12.

The inner cabinet 12 may include a frame (not shown) provided to support an upper wall 12a, a lower wall 12b, a left wall 12c, a right wall 12d, and a rear wall 12e.

The shoe care apparatus 1 may be provided to accommodate a heat pump (hereinafter referred to as a “heat exchanger”) for heating air supplied to the chamber 30 and dehumidifying air discharged from the chamber 30.

The shoe care apparatus 1 may include a machine room 40, in which a heat exchanger 47 for dehumidifying or heating air inside the shoe care room 30 and the like are provided. The machine room 40 may be disposed below the care room 30.

The shoe hanger 50 may be installed on the left wall 12c or the right wall 12d of the shoe care room 30. That is, the shoe hanger 50 may be installed such that a lateral side of a shoe is visible when viewed from the front of the shoe care apparatus 1. Accordingly, the length of the side surface of the shoe car apparatus may have a short length.

The shoe hanger 50 may be detachably installed in the shoe care room 30. The shoe hanger 50 may be provided in at least one unit thereof. The shoe hanger 50 may be formed in a shape that allows the shoe hanger 50 to be inserted into shoes.

The shoe care room 30 may be provided with a support member 80 on which the shoe hanger 50 is detachably supported. The support member 80 may be disposed on the right side of the shoe care room 30. The support member 80 may be disposed on a sidewall of the chamber 30.

The shoe hanger 50 may be detachably mounted from the support member 80. The shoe hanger 50 is provided to be mounted on the support member 80 along the front-rear direction. A specific structure of the supporting member 80 will be described below.

A water collection container 48 detachably provided from the main body 10 may be installed in a lower portion of the main body 10. The water collection container 48 may be disposed below the shoe care room 30. The water collection container 48 may be disposed below the machine room 40 to easily dispose condensation of the heat exchanger 47.

The machine room 40 may be provided on the lower side of the main body 10. The machine room 40 may be provided below the shoe care room 30. The water collection container 48 may be provided below the machine room 40.

The heat exchanger 47 may be provided to dehumidify and heat air inside the shoe care room 30 as needed.

The heat exchanger 47 may be installed to supply heated air into the shoe care room 30. The heat exchanger 47 may include an evaporator 42, a condenser 43, and a compressor 41 in which a refrigerant circulates. The heat exchanger 47 may be provided to dehumidify and heat air.

As the refrigerant evaporates in the evaporator 42 of the heat exchanger 47, the refrigerant absorbs latent heat from the surrounding air, moisture in the air may be condensed and removed.

When the refrigerant is condensed in the condenser 43 via the compressor 41, the refrigerant releases latent heat toward the surrounding air, and the surrounding air may be heated.

As the evaporator 42 and the condenser 43 perform a heat exchange function, air introduced into the machine room 40 by a fan 44 may be dehumidified and heated by sequentially passing through the evaporator 42 and the condenser 43.

The shoe care apparatus 1 may include an air supply duct 70 connecting the chamber 30 and the machine room 40. The fan 44 may be provided as a centrifugal fan that intakes air in an axial direction and discharges air toward the air supply duct 70. However, it is not limited thereto. The heat exchanger 47 installed in the machine room 40 may include a connection duct 46 connecting the evaporator 42, the condenser 43, and the fan 44, and the connection duct 46 may be connected to the shoe care room 30, forming a connection passage 46a that circulates between the shoe care room 30 and the connection duct 46.

The shoe care apparatus 1 may further include a water supply tank (not shown), a steam generating device (not shown), and a steam spraying device (not shown). The water supply tank may be disposed below the shoe care room 30. Water in the water supply tank may be supplied to the steam generating deice and used to generate steam. The water supply tank may be detachably installed from the main body to facilitate water replenishment.

The steam generating device may be provided in the machine room 40 to generate steam by receiving water from the water supply tank.

The steam generating device may be connected to the water supply tank and configured to receive water and generate steam, and include a steam supply pipe (not shown) that guides the generated steam to the steam spraying device (not shown).

The shoe care apparatus 1 may further include a deodorizing device 45. The deodorizing device 45 may be disposed within the machine room 40. The deodorizing device 45 may be disposed in the connection duct 46 to remove odors from air passed through the shoe care room 30. In the drawing, the deodorizing device 45 is illustrated as being provided on the right side of the fan 44, but it is not limited thereto and the deodorizing device 45 may be provided also on the left side of the fan 44.

The deodorizing device 45 may include a ceramic filter. However, it is not limited thereto, and the deodorizing device 45 may include various filters as long as it can remove odors in the air.

The shoe care apparatus 1 may include at least one plate 93. The plate 93 may be disposed in the shoe care room 30 to place shoes thereon. The plate 93 may be disposed underneath a respective shoe hanger 50.

The plate 93 may include a duct plate 90. The duct plate 90 may include an internal passage 90b as shown in FIG. 3. Heated air passing through the internal passage 90b may be injected toward the shoes from an injection port 90a of the duct plate 90 and an injection port 91a of a circular duct 91. In addition, heated air may be introduced into the shoe care room 30 through an inlet of the shoe hanger 50 via the internal passage 90b.

The shoe care apparatus 1 may include a support protrusion 32. The support protrusion 32 may be provided such that the plate 80 is disposed within the care room 30. The support protrusion 32 may support the plate 80 and prevent the plate 80 from falling.

The shoe care apparatus 1 may further include an air supply port 60 and a discharging port.

The air supply port 60 may be formed on a sidewall of the inner cabinet 12. That is, the air supply port 60 may be formed on the left wall 12c of the shoe care room 30. However, it is not limited thereto and the air supply port 60 may be formed on the right wall 12d.

The air supply port 60 may be formed in at least one unit thereof, and may be provided to dry shoes by supplying heated air to the inside of the shoe care room 30. The shape of the air supply port 60 may be circular. However, it is not limited thereto and the air supply port 60 may be provided in various shapes, such as a quadrangle and a polygon, as long as it can dry shoes by supplying heated air into the shoe care room 30.

The shoe care room 30 may be provided with the support member 80 that communicates with the air supply port 60. The support member 80 may be provided adjacent to the air supply port 60 on the sidewall of the chamber 30.

The support member 80 may be provided for the shoe hanger 50 to be detachably supported thereon. The support member 80 may be provided to allow the shoe hanger 50 to be detachably coupled in the front and rear directions. The support member 80 may include a support body 81 fixed to the inner cabinet 12. The support member 80 may be provided with an opening 82 formed to communicate with the air supply port 60. The opening 82 is formed to pass through the support body 81 such the shoe care room 30 and the air supply duct 70 are connected to each other. The opening 82 of the support body 81 may be opened and closed by a supply door 86.

The support member 80 may further include a shoe hanger support 84 formed to support at least a portion of the shoe hanger 50 when the shoe hanger 50 is mounted on the support member 80. The shoe hanger support 84 may be formed to protrude from the support body 81.

The support member 80 may further include a shoe hanger coupling rib 85 for guiding the shoe hanger 50 in a process of mounting or separating the shoe hanger 50 to or from the support member 80. The shoe hanger coupling rib 85 may be formed to protrude from the shoe hanger support 84. The shoe hanger coupling rib 85 may extend along a direction in which the shoe hanger 50 is mouthed to or separated from the support member 80.

The discharging port 31 may be disposed in a front portion of the lower wall 12b of the shoe care room 30. However, the location of the discharging port 31 is not limited thereto. Air having been used to dry shoes in the shoe care room 30 may flow through the discharging port 31 to the connection duct 46 to be described below.

The discharging port 31 may be provided such that heated air may be smoothly supplied to the connection duct 46.

The connection duct 46 may be connected to the air supply duct 70 and the discharging port 31 of the shoe care room 30. One end of the connection duct 46 may be connected to the air supply duct 70, and the other end of the connection duct 46 may be connected to the discharging port 31. The air introduced through the discharging port 31 may be dehumidified and introduced back into the shoe care room 30 through the air supply duct 70 and the air supply port 60.

One end of the air supply duct 70 may be connected to at least one air supply port 60, and the other end of the air supply duct 70 may be connected to the connection duct 46. The air supply duct 70 may form a supply passage 71 provided to move heated air toward the air supply port 60.

The shoe care apparatus 1 may further include the shoe hanger 50.

The shoe hanger 50 may be detachably mounted on the inner surface of the sidewall of the inner cabinet 12. That is, the shoe hanger may be coupled to the left wall 12c of the shoe care room 30, or the right wall 12d of the shoe care room 30. The shoe hanger 50 may be disposed on the right wall 12d of the shoe care room 30 as long as it can be supplied with heated air.

A detailed description of the shoe hanger 50 will be described below.

The shoe care apparatus 1 may further include a sterilization device 100. The sterilization device 100 may be disposed inside the shoe care room 30. The sterilization device 100 may be provided to sterilize shoes S accommodated in the shoe care room 30 by being hung on the shoe hanger 50. The sterilization device 100 may be provided to sterilize the outer and inner surfaces of the shoes S. The sterilization device 100 may be installed inside the shoe care room 30 to sterilize the exterior of the shoe S. The sterilization device 100 may be installed inside the shoe care room 30 to sterilize the interior of the shoe S. The sterilization device 100 may be provided inside a hollow of the shoe hanger 50.

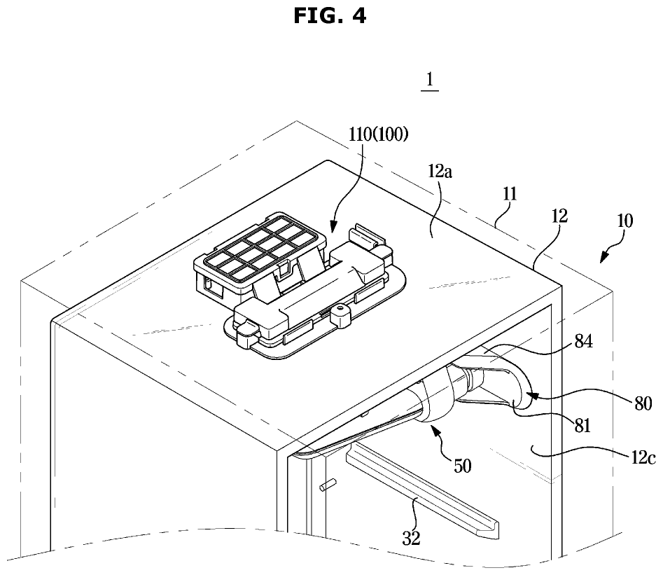

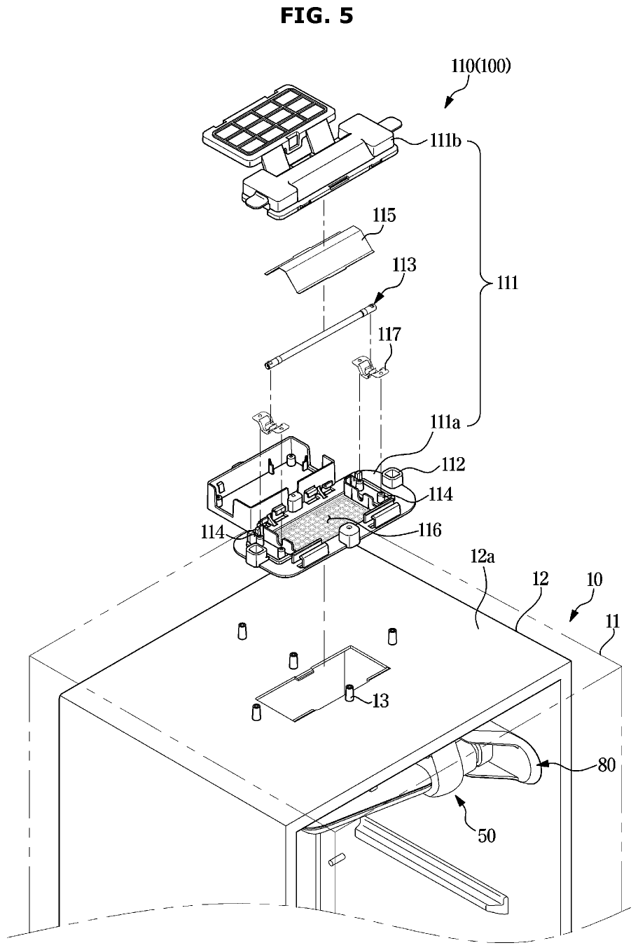

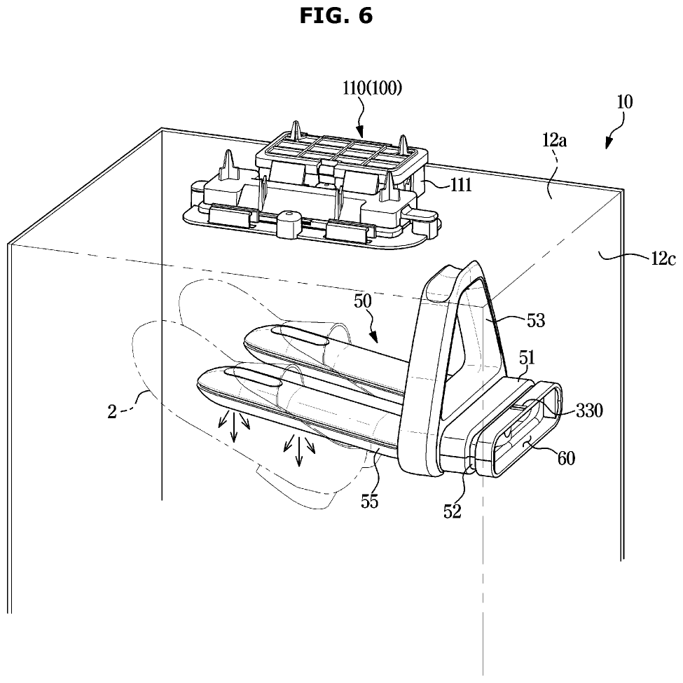

FIG. 4 is a perspective view illustrating components related to a sterilization device of the shoe care apparatus shown in FIG. 3. FIG. 5 is an exploded view illustrating a first sterilization portion of the shoe care apparatus shown in FIG. 4. FIG. 6 is a perspective view illustrating a second sterilization portion mounted on a shoe hanger shown in FIG. 4.

Referring to FIGS. 4 to 6, the shoe care apparatus 1 may further include the sterilization device 100 for sterilizing shoes S.

The sterilization device 100 may include a first sterilization portion 110 installed on the inner cabinet 12 of the shoe care room 30 and a second sterilization portion 120 installed in the shoe hanger 50. The sterilization device 100 may include the first sterilization portion 110 provided in the shoe care room 30 to sterilize the exterior of the shoe S. The sterilization device 100 may include the second sterilization portion 120 provided in the shoe hanger 50 to sterilize the interior of the shoe S.

The first sterilization portion 110 of the sterilization device 100 may be installed on the upper wall 12a of the inner cabinet 12. The first sterilization portion 110 may be disposed between the outer cabinet 11 and the inner cabinet 12. The first sterilization portion 110 may not be externally exposed to the outside and may improve aesthetics of the shoe care apparatus.

The first sterilization portion 110 is provided to sterilize the exterior of the shoe S mounted on the shoe hanger 50. Although the first sterilization portion 110 provided to sterilize the exterior of the shoes S is illustrated as being disposed on the upper wall 12a, the concept of the disclosure is not limited thereto. For example, the first sterilization portion 110 may be disposed on the left wall 12c or the right wall 12d. In addition, the first sterilization portion 110 may be disposed on the rear wall 12e or may be disposed on the door. The sterilization device 100 may be installed on any other wall than a wall in which the shoe hanger 50 is installed. The sterilization device 100 may be placed in various positions within the shoe care apparatus, enable flexible utilization of space in the shoe care apparatus.

The first sterilization portion 110 may include a case 111, an ultraviolet (UV) lamp 113, a reflective member 115, and a bracket 117. The case 111 may be provided to accommodate the UV lamp 113, the reflective member 115, and the bracket 117 therein.

The case 111 may include a first case 111a and a second case 111b. The first case 111a may be disposed on the lower side, and the second case 111b may be disposed on the upper side.

The first case 111a may be provided to be coupled to the upper wall 12a. The first case 111a may be provided to be coupled to a coupling protrusion 13 on the upper wall 12a. The first case 111a may include an installation portion 116 in which the UV lamp 113 is installed and accommodated, a coupling portion 112 configured to be coupled to the coupling protrusion 13, and a bracket coupling portion 114 allowing the bracket 117 to be installed thereon.

The second case 111b may be coupled to the first case 111a to accommodate the UV lamp 113, the reflective member 115, and the like. The second case 111b may be coupled to the first case 111a from the upper side.

The UV lamp 113 may be disposed in the case 111 to emit ultraviolet rays for sterilizing the shoes S. The UV lamp 113 may be provided in a cylindrical shape. In the embodiment of the present disclosure, the UV lamp has a cylindrical shape, but it is not limited thereto. The UV lamp 113 may emit Ultraviolet-C (UVC). However, it is not limited thereto, and the UV lamp 113 may also emit Ultraviolet-A (UVA) and Ultraviolet-B (UVB).

The reflective member 115 may reflect ultraviolet rays emitted from the UV lamp 113. The reflective member 115 may be disposed above the UV lamp 113. Accordingly, ultraviolet rays directed upward may be reflected downward through the reflective member 115, allowing ultraviolet rays of the UV lamp 113 to be directed only to the shoes.

As described above, the ultraviolet rays emitted from the UV lamp 113 of the first sterilization portion 110 may sterilize the exterior of the shoes S placed in the shoe care room 30.

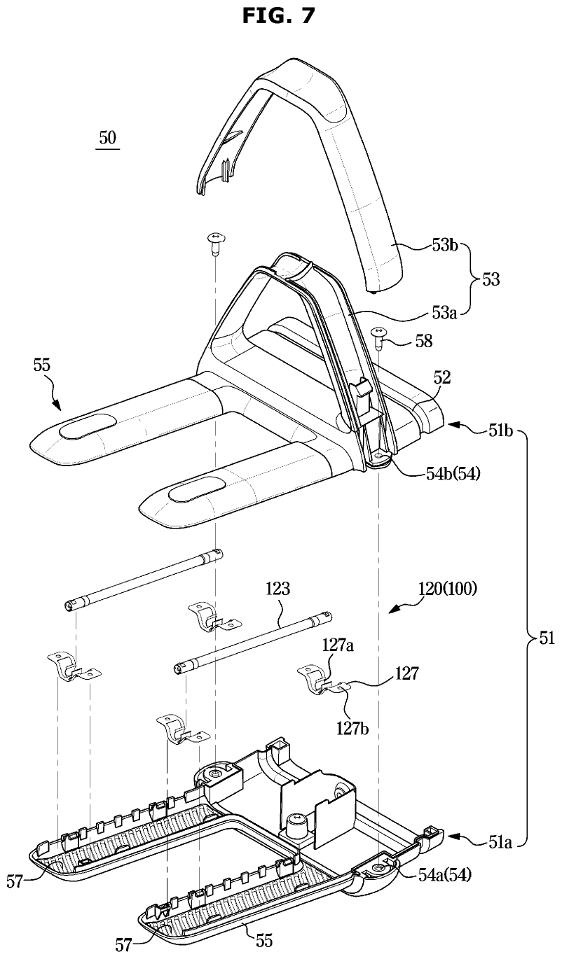

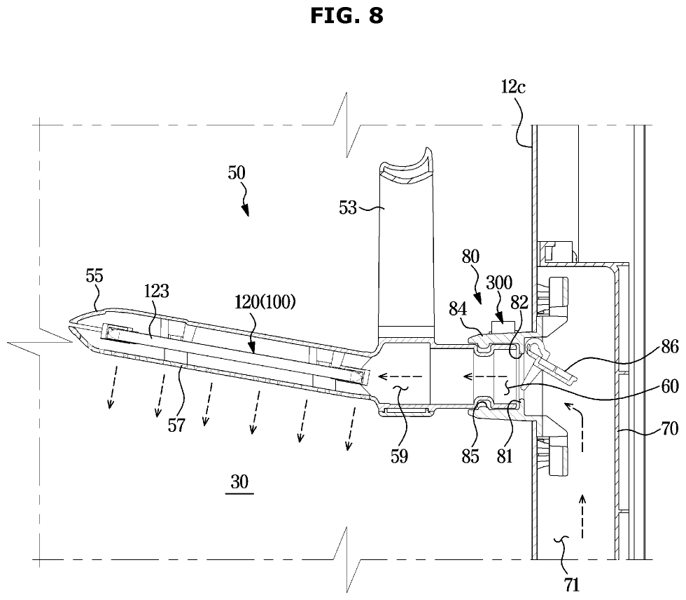

FIG. 7 is an exploded perspective view illustrating a second sterilization portion mounted on the shoe hanger shown in FIG. 6, and FIG. 8 is a cross-sectional view illustrating a second sterilization portion mounted on the shoe hanger shown in FIG. 6.

Referring to FIGS. 7 to 8, the sterilization device 100 may include the second sterilization portion 120 provided in the shoe hanger 50 to sterilize the interior of the shoe S.

The second sterilization portion 120 of the sterilization device 100 may be installed in the shoe hanger 50. The second sterilization portion 120 may be installed inside the shoe hanger 50. The shoe hanger 50 may be provided with a hollow formed therein. The sterilization device 100 may be provided inside the hollow of the shoe hanger 50. The second sterilization portion 120 may be provided inside the hollow of the shoe hanger 50.

The second sterilization portion 120 may not be exposed externally, and thus aesthetics of the shoe care apparatus 1 may be improved.

The second sterilization portion 120 is provided to sterilize the inside of the shoes S mounted on the shoe hanger 50. The second sterilization portion 120 is provided to sterilize the interior of the shoe S mounted on the shoe hanger 50.

The shoe hanger 50 in which the second sterilization portion 120 is installed may include a shoe hanger body 51 forming the external appearance, a passage 59 formed inside the shoe hanger body 51, and a plurality of holes 57 formed through the shoe hanger body 51 in connection with the passage 50 to supply air into the shoe care room 30. The plurality of holes 57 may be formed on a lower surface of the shoe hanger body 51.

The shoe hanger body 51 may include a first body 51a forming a lower portion and a second body 51b forming an upper portion. The first body 51a and the second body 51b may be coupled to each other to form the shoe hanger body 51. The passage 59 may be formed between the first body 51a and the second body 51b. The first body 51a and the second body 51b may be formed in corresponding shapes, and the passage 59 is formed in a space formed by the first body 51a and the second body 51b coupled to each other. The first body 51a and the second body 51b may form an upper surface and a lower surface of the shoe hanger body 51, respectively.

The first body 51a and the second body 51b may be coupled to each other by a fixing portion 54. The fixing portion 54 may include a first fixing portion 54a formed on the first body 51a and a second fixing portion 54b formed on the second body 51b. The first fixing portion 54a and the second fixing portion 54b are provided at positions corresponding to each other, and may be fixed by a fixing member 58 passing through the first fixing portion 54a and the second fixing portion 54b.

The passage 59 formed inside the shoe hanger body 51 is provided to allow air introduced from the air supply duct 70 to flow therein. The passage 59 formed inside the first body 51a and the second body 51b of the shoe hanger body 51 is provided to allow cooling air passed through the fan 44 and the air supply duct 70 to flow therein. In order for the air moving through the passage 59 of the shoe hanger body 51 to be injected into the shoe care room 30, the shoe hanger body 51 may be formed with a plurality of holes 57. The plurality of holes 57 may be formed in the first body 51a forming the lower surface of the shoe hanger body 51. Cooling air passed through the fan 44 and the air supply duct 70 may be provided to pass through the passage 59 of the shoe hanger 50 and then be injected into the shoe care room 30 through the plurality of holes 57.

The shoe hanger 50 may include a pair of support arms 55 formed to protrude such that the shoes S may be supported thereon. The support arm 55 may be formed to extend in the first direction X of the shoe hanger body 51. Two support arms 55 may be formed as a pair. The shoe hanger 50 may be provided for a pair of shoes S to be supported by the pair of support arms 55. The plurality of holes 57 may be formed on the bottom surface of the pair of support arms 55.

The shoe hanger 50 may further include a handle 53 provided for the user to move the shoe hanger 50 after placing the shoes S on the pair of support arms 55. The handle 53 may be provided on the upper side of the shoe hanger body 51. The handle 53 may be provided on the second body 51b forming the upper surface of the shoe hanger body 51. The handle 53 may include a first handle portion 53a extending upwardly from the second body 51b and a second handle portion 53b coupled to the first handle portion 53a from the upper side. In the embodiment of the present disclosure, the handle is illustrated as including the first handle portion and the second handle portion separately provided and coupled to each other, but the concept of the present disclosure is not limited thereto. For example, the handle may be integrally formed with the shoe hanger body.

In the passage 59 formed inside the shoe hanger 50, the second sterilization portion 120 may be disposed. The second sterilization portion 120 may be disposed on the passage 59.

The second sterilization portion 120 may include an UV lamp 123 and an installation bracket 127. The second sterilization portion 120 may be installed inside the shoe hanger 50. The second sterilization portion 120 may be disposed inside the pair of support arms 55 of the shoe hanger 50. The second sterilization portion 120 may be disposed inside the pair of support arms 55 of the shoe hanger 50 and configured to sterilize the inside of the shoes S supported by the pair of support arms 55.

The shoe hanger 50 may include the shoe hanger body 51. The shoe hanger body 51 may include a pair of support arms 55 on each of the first body 51a and the second body 51b.

The second sterilization portion 120 may further include the installation bracket 127 for mounting the UV lamp 123 thereon such that the UV lamp 123 may be positioned on the passage 59 of the shoe hanger body 51.

The UV lamp 123 is disposed within the pair of support arms 55, and is configured to emit ultraviolet rays for sterilizing the interior of the shoes S. The UV lamp 123 may be provided in a cylindrical shape. In the embodiment of the present disclosure, the UV lamp has a cylindrical shape, but it is not limited thereto. The UV lamp 123 may emit UVC. However, it is not limited thereto, and the UV lamp 123 may also emit UVA and UVB.

The installation bracket 127 may be provided as two installation brackets 127 as a pair to support one side and the other side of the UV lamp 123. The installation bracket 127 includes an UV lamp support portion 127a formed in a shape corresponding to the UV lamp 123 to support the UV lamp 123 and an installation bracket fixing portion 127b formed to fix the installation bracket 127 to the second body 51b. The installation bracket 127 may be fixed to the second body 51b of the shoe hanger body 51 to position the UV lamp 123 on the passage 59.

The second sterilization portion 120, i.e., the UV lamp 123, disposed on the pair of support arms 55 of the shoe hanger 50, may allow the inside of the shoes S supported on the shoe hanger 50 to be sterilized as a whole.

In addition, as the cooling air passed through the fan 44 and the air supply duct 70 flows through the passage 59 of the shoe hanger 50, the UV lamp 123 disposed on the passage 59 always contacts the cooling air, thus suppressing a temperature rise of the UV lamp 123 during the sterilization operation and preventing damage to the shoes S due to overheating.

The shoe hanger 50 may further include a power connection part 330 for supplying power to the second sterilization portion 120 installed in the passage 59. The power connection part 330 may be connected to the power supply part 300 provided on the support member 80 to apply power to the second sterilization portion 120.

When the shoe hanger 50 is mounted on the support member 80, the power connection part 330 may be provided to be connected to the power supply part 300 provided on the support member 80.

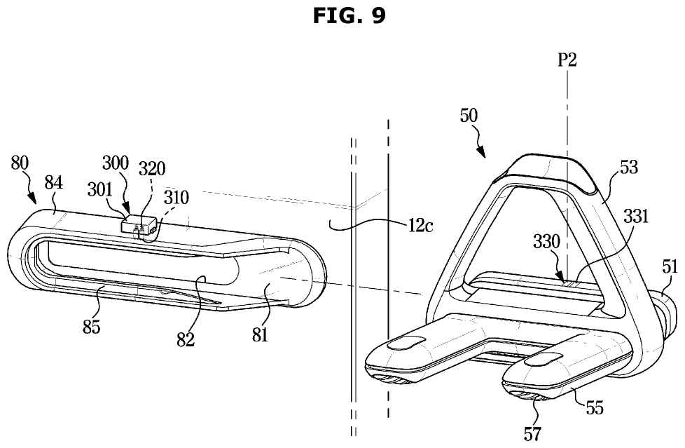

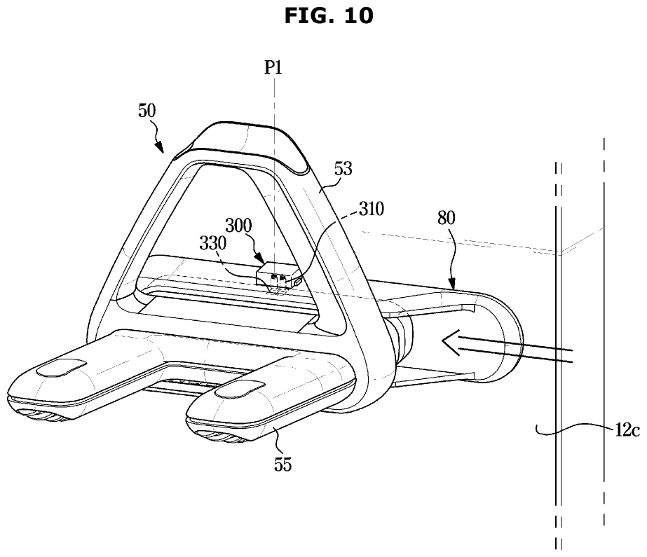

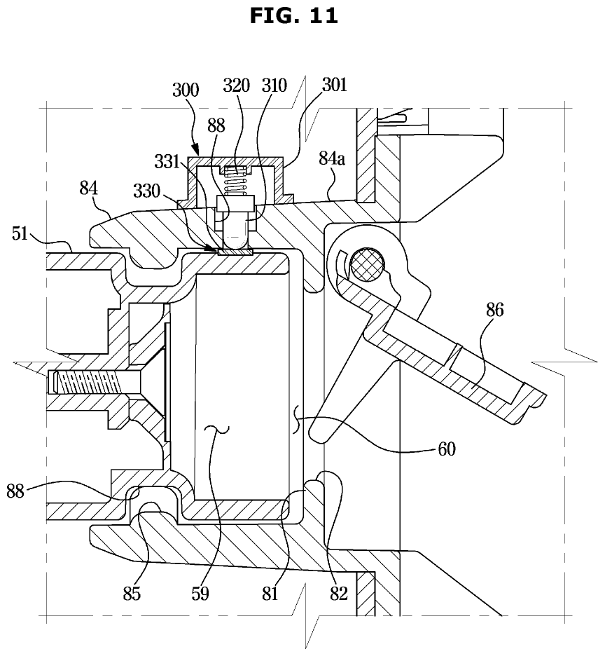

FIG. 9 is a view illustrating a state in which the shoe hanger shown in FIG. 4 is being mounted on a support member, FIG. 10 is a view illustrating the shoe hanger shown in FIG. 4 mounted on the support member, which shows a state in which a power connection part is being connected to a power supply part, and FIG. 11 is a cross-sectional view illustrating a coupled state of the power supply part and the power connection part shown in FIG. 10.

Referring to FIGS. 9 to 11, the shoe hanger 50 may further include the power connection part 330 for supplying power to the second sterilization portion 120 installed in the passage 59.

The power connection part 330 may be connected to the power supply part 300 provided on the support member 80 to apply power to the second sterilization portion 120. The power connection part 330 may include a connection terminal 331.

The power connection part 330 may be provided on the shoe hanger body 51 of the shoe hanger 50. The power connection part 330 may be disposed on the upper surface of the shoe hanger body 51.

The power connection part 330 of the shoe hanger 50 may be that corresponds to the power supply part 300 of the support member 80 so as to be connected to the power supply part 300 of the support member 80.

The power supply part 300 may include a contact terminal 310 provided to be in contact with the power connection part 330. The power supply part 300 may include the contact terminal 310 provided to be in contact with the connection terminal 331 of the power connection part 330.

The power supply part 300 may further include a power case 301 provided to accommodate the contact terminal 310 and an elastic body 320 provided to elastically support the contact terminal 310 inside the power case 301.

The elastic body 320 mounted inside the power case 301 is provided to elastically support the contact terminal 310. The elastic body 320 may elastically support the contact terminal 310 for the contact terminal 310 to be movable in a direction perpendicular to the mounting direction of the shoe hanger 50 by a movement of the shoe hanger 50.

The power supply part 300 may be provided on the support member 80. The power supply part 300 may be disposed on the upper portion of the support member 80. The support member 80 may include the shoe hanger support 84 protruding from the support body 81 to support the shoe hanger body 51. The shoe hanger support 84 may include a first support 84a forming an upper part, a second support 84b forming a side part, and a third support 84c forming a lower part.

The first support 84a and the third support 84c may be positioned to correspond to each other while being spaced apart from each other by a predetermined interval. The second support 84b may be provided to connect one side of the first support 84a and one side of the third support 84c. The first support 84a may be provided to support the upper surface of the shoe hanger body 51, the third support 84c may be provided to support the lower surface of the shoe hanger body 51, and the second support 84b may be provided to support one surface of the shoe hanger body 51.

The power supply part 300 may be disposed on the first support 84a arranged on the upper side of the shoe hanger support 84. A terminal installation portion 88 for installing the power supply part 300 may be formed on the first support 84a. The terminal installation portion 88 may be formed to pass through the first support 84a such that the contact terminal 310 of the terminal installation portion 88 passes through the first support 84a so as to be in contact with the connection terminal 331 provided on the shoe hanger 50.

Therefore, in a first position P1 of the shoe hanger 50 being coupled to the support member 80 and mounted, the power connection part 330 of the shoe hanger 50 is connected to the power supply part 300 of the support member 80, and thus the UV lamp 123 of the second sterilization portion 120 is powered on.

The power connection part 330 and the power supply part 300 may check the application of power by a contact between the connection terminal 331 and the contact terminal 310, at the same time as checking whether the UV lamp 123 is operating in a normal state or operating in an abnormal state.

When the shoe hanger 50 is completely mounted in the mounting position of the support member 80, the power connection part 330 and the power supply part 300 are connected to each other and thus the UV lamp 123 is powered on, with which it may be checked whether the shoe hanger 50 is mounted at a mounting position of the support member 80.

In addition, in a second position P2 of the shoe hanger 50 being separated from the support member 80, the power connection part 330 of the shoe hanger 50 and the power supply part 300 of the support member 80 are separated from each other and thus the UV lamp 123 is powered off

The power connection part 330 and the power supply part 300 may allow for checking the application of power by the connection between the connection terminal 331 and the contact terminal 310 provided on the shoe hanger 50 and the support member 80, respectively, and when the shoe hanger 50 is separated from the support member 80, the power connection part 330 and the power supply part 300 are separated from each other, and the UV lamp 123 is powered off.

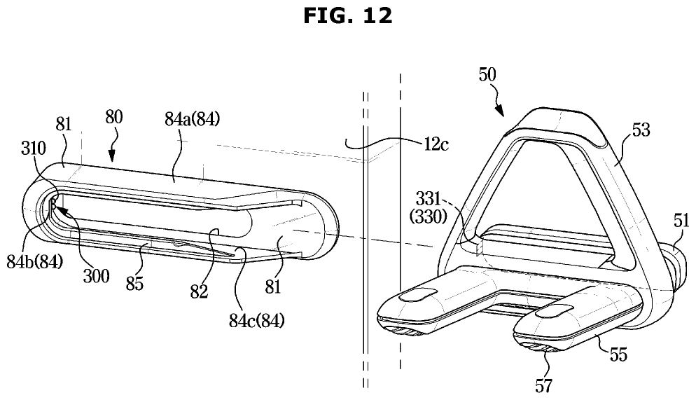

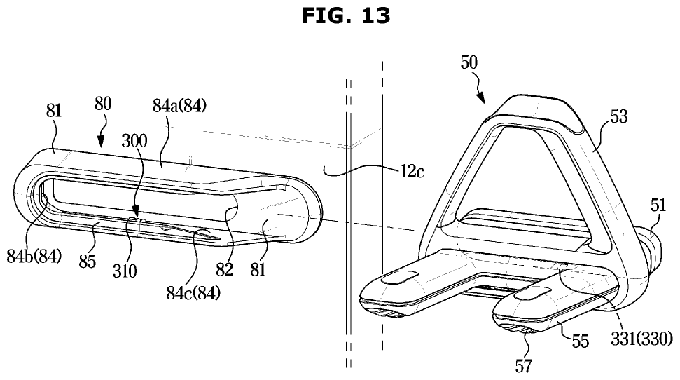

FIGS. 12 and 13 are diagrams illustrating a power supply part and a power connection part mounted in a different position according to another embodiment of the present disclosure.

Referring to FIG. 12, the shoe hanger 50 may further include a power connection part 330 for supplying power to the second sterilization portion 120 installed in the passage 59. The power connection part 330 may be connected to the power supply part 300 provided on the support member 80 to apply power to the second sterilization portion 120. The power connection part 330 may include the connection terminal 331.

The power connection part 330 may be provided on the shoe hanger body 51 of the shoe hanger 50. The power connection part 330 may be disposed on the side surface of the shoe hanger body 51, but is not limited thereto.

The power connection part 330 may even serve as a sensing device for detecting whether the shoe hanger 50 is mounted in response to the shoe hanger 50 being slidably coupled to the support member 80 and mounted,

The power connection part 330 may be disposed on one side surface of the shoe hanger 50 to check whether the shoe hanger 50 is completely seated on the support member 80.

The power supply part 300 corresponding to the power connection part 330 may be provided on the support member 80.

The power supply part 300 may be disposed on the second support 84b disposed on the lateral side of the shoe hanger support 84. A terminal installation portion (not shown) for installing the power supply part 300 may be formed in the second support 84b. The terminal installation portion may be formed to pass through the second support 84b such that the contact terminal 310 may directly contact the connection terminal 331 of the shoe hanger 50.

In a first position P1 of the shoe hanger 50 being coupled to the support member 80 and completely mounted, the power connection part 330 of the shoe hanger 50 and the power supply part 300 of the support member 80 are connected to each other, and the UV lamp 123 of the second sterilization portion 120 is powered on to thereby operate.

The power connection part 330 and the power supply part 300 may check the application of power by a contact between the connection terminal 331 and the contact terminal 310, and at the same time as checking whether the UV lamp 123 is abnormal.

Referring to FIG. 13, the power connection part 330 of the shoe hanger 50 is connected to the power supply part 300 provided on the support member 80 to supply power to the second sterilization portion 120.

The power connection part 330 may be provided on the shoe hanger body 51 of the shoe hanger 50. The power connection part 330 may be disposed on the lower surface of the shoe hanger body 51, but is not limited thereto.

The power connection part 330 may even serve as a sensing device for detecting whether the shoe hanger 50 is mounted in response to the shoe hanger 50 being slidably coupled to the support member 80 and mounted,

The power connection part 330 is disposed on the lower surface of the shoe hanger 50. The power supply part 300 may be disposed on the third support 84c forming the lower part of the shoe hanger support 84.

The power connection part 330 and the power supply part 300 may be provided to check whether the shoe hanger 50 is seated at the correct position of the support member 80.

In a first position P1 of the shoe hanger 50 being mounted on the support member 80, the power connection part 330 of the shoe hanger 50 is connected to the power supply part 300 of the support member 80, and thus the UV lamp 123 of the second sterilization portion 120 may be powered on.

Shoes S are mounted on the support member 80 while being placed on the shoe hanger 50. As the power connection part 330 and the power supply part 300 are disposed on the lower side of the shoe hanger 50 and on the lower side of the support member 80, the load of the shoes S applied to the shoe hanger 50 may enable the physical contact between the connection terminal 331 and the contact terminal 310 to be firmly secured.

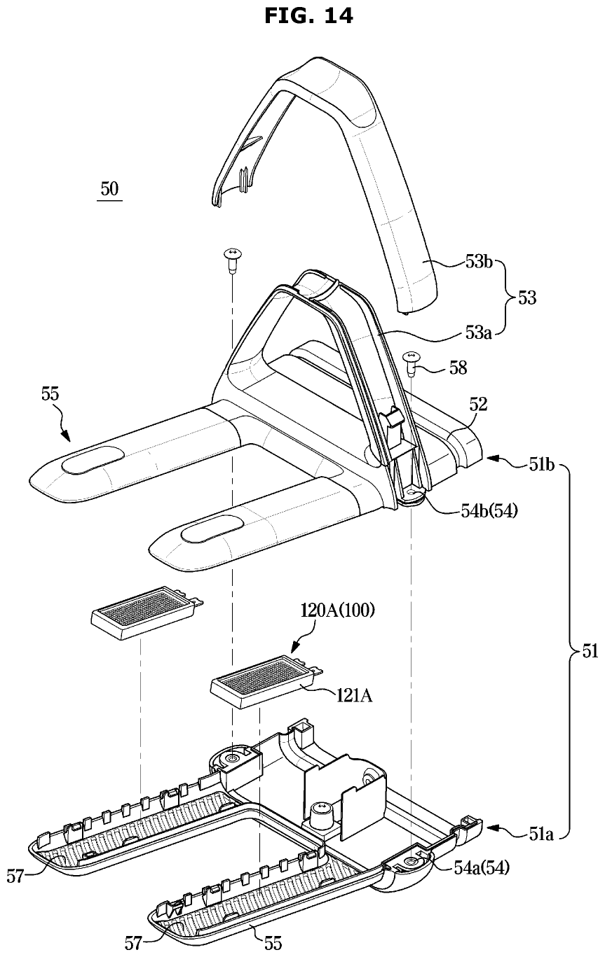

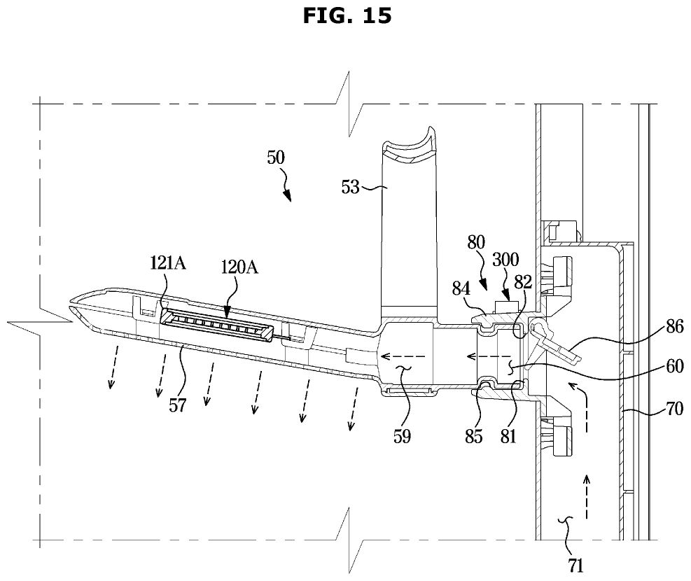

FIG. 14 is an exploded perspective view illustrating a shoe hanger with a second sterilization portion mounted thereon according to another embodiment of the present disclosure. FIG. 15 is a cross-sectional view illustrating the second sterilization portion mounted on the shoe hanger shown in FIG. 14.

Referring to FIGS. 14 and 15, the sterilization device 100 may include a second sterilization portion 120A provided on the shoe hanger 50 to sterilize the interior of the shoe S.

The second sterilization portion 120A is provided to sterilize the inside of the shoes S hung on the shoe hanger 50.

The second sterilization portion 120A may be installed on the shoe hanger 50. The second sterilization portion 120A may be installed inside the shoe hanger 50. The second sterilization portion 120A may not be externally exposed, and thus aesthetics of the shoe care apparatus 1 may be improved.

The second sterilization portion 120A may include an ozone generator 121A and an installation bracket 127A on which the ion generator 121A is installed.

The ozone generator 121A of the second sterilization portion 120A is provided to sterilize the inside of the shoe S by generating ozone. The ozone generator 121A may perform sterilization by radicals generated by plasma discharge in the direction of air flow.

The ozone generator 121A may be installed in the passage 59 inside the shoe hanger 50 by being supported by the installation bracket 127A. The ozone generator 121A may be disposed inside the pair of support arms 55 of the shoe hanger 50 to sterilize the inside of the shoes S supported by the pair of support arms 55.

In the embodiment of the present disclosure, the ozone generator 121A is illustrated as a rectangular shape, but the concept of the present disclosure is not limited thereto.

The installation bracket 127A may be provided as two installation brackets 127A as a pair to support one side and the other side of the ozone generator 121A. The installation bracket 127A may be provided to position the ozone generator 121A on the passage 59.

The second sterilization portion 120A, i.e., the ozone generator 121A, disposed in the pair of support arms 55 of the shoe hanger 50 may sterilize the inside of the shoes S supported by the shoe hanger 50.

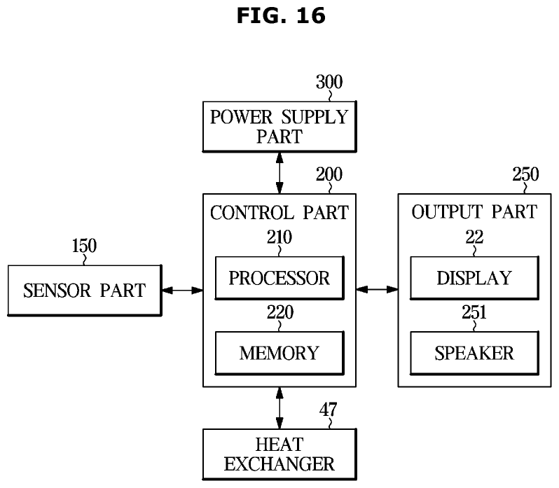

FIG. 16 is a control block diagram illustrating a shoe care apparatus according to an embodiment of the present disclosure.

Referring to FIG. 16, the shoe care apparatus 1 according to an embodiment may include a sensor part 150, a control part 200, an output part 250, a power supply part 300, and a heat exchanger 47.

The heat exchanger 47 may include the evaporator 42, the condenser 43, and the compressor 41 in which a refrigerant circulates, and may be provided to dehumidify and heat air introduced from the chamber.

The heat exchanger 47 may transmit operating information (e.g., the operating frequency of the compressor 41) to the control part 200, and the control part 200 may control the heat exchanger 47 (e.g., the compressor 41).

The sensor part 150 may include various sensors for obtaining information about each component of the shoe care apparatus 1 or the internal environment of the shoe care apparatus 1.

According to various embodiments, the sensor part 150 may include a temperature sensor (not shown) configured to sense the temperature at each location (e.g., at the air supply duct 70) of the shoe care apparatus 1.

In one embodiment, the sensor part 150 may include a sensor (e.g., a current sensor and a voltage sensor) configured to obtain various information for identifying whether the power supply part 300 supplies power to the sterilization device 100.

The sensor part 150 may transmit information (e.g., information about a power supply state of the power supply part 300) about each component of the shoe care apparatus 1 or the internal environment of the shoe care apparatus 1 to the control part 200.

The output part 250 is a component configured to provide various information to a user, and may include a display 22 and/or a speaker 251.

The display 22 may output a visual indication to provide the user with a variety of information, and the speaker 251 may output a sound to provide the user with a variety of information.

In one embodiment, the display 22 may include a touch screen configured to receive various commands (hereinafter a “user input”) from a user.

According to various embodiments, the shoe care apparatus 1 may include an input device (not shown) for receiving a user input, separately from the display 22. The input device (not shown) may be implemented in various forms (e.g., buttons, touch screen, etc.) for receiving a user input.

The power supply part 300 may be connected to the power connection part 330 in a state in which the shoe hanger 50 is mounted on the support member 80, to supply the sterilization device 100 with power.

The control part 200 may include at least one processor 210 and at least one memory 220 and may control at least one other component of the shoe care apparatus 1.

The processor 210 may execute software (e.g., a program) to control at least one other component (e.g., the heat exchanger 47, the output part 250, the fan 44, the sterilization device 100, and the power supply part 300) of the shoe care apparatus 1 connected to the processor 210, and to perform various data processing or calculations. According to one embodiment, as at least part of the data processing or calculation, the processor 210 may store instructions or data received from other components (e.g., the sensor part 150, the display 22, and/or the input device (not shown)) in a volatile memory, process the instructions or data stored in the volatile memory, and store resultant data in a non-volatile memory. According to one embodiment, the processor 210 may include a main processor (e.g., a central processing unit) or an auxiliary processor (e.g., a processor for controlling a heat exchanger and a processor for controlling an output part) that may be operated independently from or together with the main processor. For example, when the processor 210 includes a main processor and an auxiliary processor, the auxiliary processor may use less power than the main processor or may be set to be specialized for a designated function. The auxiliary processor may be implemented separately from or as part of, the main processor.

The memory 220 may store various data used by at least one component (e.g., the processor 210 or the sensor part 150) of the shoe care apparatus 1. The data may include, for example, software and input data or output data regarding software related instructions. The memory 220 may include a volatile memory or a non-volatile memory.

According to various embodiments, the memory 220 may store software (e.g., a program) that, when executed by the processor 210, performs an operation described below.

In the above description, the configuration of the shoe care apparatus 1 according to various embodiments has been described above, and hereinafter, a method of controlling a shoe care apparatus 1 according to various embodiments will be described in detail below.

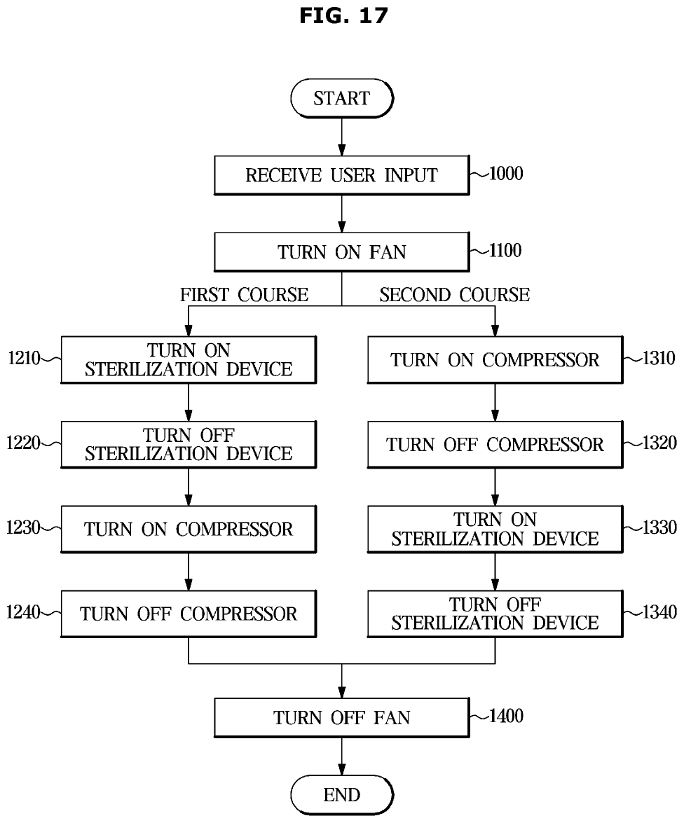

FIG. 17 is a flowchart showing an example of course operations of a shoe care apparatus according to an embodiment of the present disclosure.

Referring to FIG. 17, the input device (e.g., a touch screen of the display 22) of the shoe care apparatus 1 may receive a user input for starting various courses (e.g., a first course and a second course) of the shoe care apparatus 1 (1000).

The control part 200 may, based on receiving a user input for starting a course of the shoe care apparatus 1, turn on the fan 44 (1100).

In one embodiment, the control part 200 may alternately turn on the compressor 41 and the sterilization device 100.

Specifically, the control part 200 may control the compressor 41 and the sterilization device 100 such that the on-time of the compressor 41 and the on-time of the sterilization device 100 do not overlap each other.

According to various embodiments, the control part 200 may control the power supply part 300 to cut off power applied to the sterilization device 100 to turn off the sterilization device 100.

In one embodiment, the control part 200 may turn on the sterilization device 100 based on the compressor 41 being turned off, and turn on the compressor 41 based on the sterilization device 100 being turned off

Specifically, the control part 200 may, based on receiving a user input for starting the first course, turn on the sterilization device 100 for a preset time corresponding to the first course (1210), and upon the preset time elapsing, turn off the sterilization device 100 (1220).

The control part 200 may, based on the sterilization device 100 being turned off, turn on the compressor 41 for a preset time corresponding to the first course (1230), and upon the preset time elapsing, turn off the compressor 41 (1240).

According to various embodiments, the control part 200 may, based on the compressor 41 being turned off, turn on the sterilization device 100 again.

Thereafter, the control part 200 may, based on the course being completed, turn off the fan 44 (1400).

In another embodiment, the control part 200 may, based on receiving a user input for starting the second course, turn on the compressor 41 for a preset time corresponding to the second course (1310), and upon the preset time elapsing, turn off the compressor 41 (1320).

The control part 200 may, based on the compressor 41 being turned off, turn on the sterilization device 100 for a preset time corresponding to the second course (1330), and upon the preset time elapsing, turn off the sterilization device 100 (1340).

In one embodiment, the control part 200 may turn on the sterilization device 100 upon a preset period elapsing since the compressor 41 is turned off

Thereafter, the control part 200 may, based on the course being completed, turn off the fan 44 (1400).

The shoe care apparatus 1 according to an embodiment may operate the compressor 41 and the sterilization device 100 without overlapping each other, thereby preventing the sterilization device 100 from being damaged by hot air.

Although not shown in the drawings, according to various embodiments, the control part 200 may control on/off of the sterilization device 100 based on temperature information obtained from the sensor part 150.

For example, in order to prevent damage to the sterilization device 100 due to high temperature, the control part 200 may turn on the sterilization device 100 only when the temperature inside the shoe care apparatus 1 (e.g., the temperature of the air supply duct 70) is less than or equal to a preset temperature.

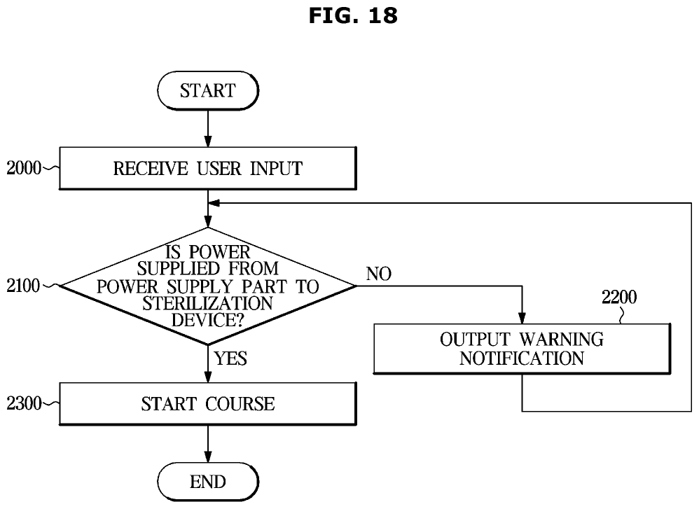

FIG. 18 is a flowchart illustrating an example of outputting a warning notification by a shoe care apparatus 1 according to an embodiment of the present disclosure.

Referring to FIG. 18, the input device (e.g., the touch screen of the display 22) of the shoe care apparatus 1 may receive a user input for starting various courses of the shoe care apparatus 1 (2000).

The control part 200 may identify whether the power supply part 300 is supplying power to the sterilization device based on information (e.g., the power supply state of the power supply part 300) obtained through the sensor part 150 (2100).

According to various embodiments, the control part 200 may, in a case of no current flowing from the power supply part 300 to the power connection part 330, identify that power is not applied to the sterilization device through the power supply part 300, and in a case of a current greater than or equal to a preset value flowing from the power supply part 300 to the power connection part 330, identify that power is applied to the sterilization device through the power supply part 300.

In various embodiments, the control part 200 may, based on the sterilization device 100 not being turned on even by outputting a control signal for turning on the sterilization device 100 a preset number of times, identify that power is not applied to the sterilization device through the power supply part 300.

A method of identifying whether the power supply part 300 is providing power to the sterilization device 100 is not limited to the above embodiment, and various methods within the general technical range may be used.

The control part 200 may, based on identifying that no power is applied to the sterilization device 100 through the power supply part 300, control the output part 250 to output a warning notification (e.g., a visual notification or an audible notification) notifying that the shoe hanger 50 is separated from the support member 80.

For example, the display 22 may display a visual notification (e.g., a phrase, a figure, an icon, etc.) notifying that the shoe hanger 50 is separated from the support member based on a control signal from the control part 200.

As another example, the speaker 251 may output a sound e.g., a guide speech, a buzzer sound, etc.) notifying that the shoe hanger 50 is separated from the support member based on a control signal from the control part 200.

A user may then reattach the shoe hanger 50 to the support member 80 to reconnect the shoe hanger 50 and the support member 80, to allow power to be applied to the sterilization device 100.

The control part 200 may, based on identifying that a power is applied to the sterilization device 100 through the power supply part 300, control each component (e.g., the fan 44, the heat exchanger 47, and the sterilization device 100) of the shoe care apparatus 1 to start a course corresponding to a user input (2300).

The shoe care apparatus 1 according to an embodiment may identify whether the shoe hanger 50 is properly mounted to the support member 80 based on whether power is applied to the sterilization device 100, and in a case of the support member 50 not being properly mounted on the support member 80, output a warning notification to allow the user to properly mount the shoe hanger 50.

In various embodiments, the control part 200 may, in response to receiving a user input for starting a course of the shoe care apparatus 1, identify whether power is applied to the sterilization device 100 through the power supply part 300, and based on the result, output a warning notification, but may identify whether power is applied to the sterilization device 100 through the power supply part 300 at preset time periods regardless of whether a user input is received, and output a warning notification according to the result.

According to various embodiments, the power supply part 300 may be provided on the upper inner wall of the support member 80, and in this case, the power connection part 330 may be designed not to be connected to the power supply part 300 even when the shoe hanger 50 is properly mounted on the support member 80.

Specifically, the power connection part 330 may be designed to be connected to the power supply part 300 only when the shoe hanger 50 is properly mounted on the support member 80 and then shoes S are placed on the shoe hanger 50 and a load of the shoes S on the shoe hanger 50 generates a torque.

In this case, the control part 200 may, based on identifying that power is not being applied to the sterilization device 100 through the power supply part 300, control the output part 250 to output a warning notification (a visual notification or an auditory notification) notifying that the shoes S are not hung on the shoe hanger 50.

The shoe care apparatus 1 according to an embodiment may identify whether the shoe hanger 50 is properly mounted to the support member 80 and also whether shoes S are placed on the shoe hanger 50 based on the power supply state of the power supply part 300 according to various designs, and upon identifying that the shoe hanger 50 is not properly mounted on the support member 80 or shoes S are not placed on the shoe hanger 50, output a warning notification, thereby allowing the user to properly mount the shoe hanger 50 or place shoes S on the shoe hanger 50.

Although the disclosure has been shown and described in relation to specific embodiments, it would be appreciated by those skilled in the art that changes and modifications may be made in these embodiments without departing from the principles and scope of the disclosure, the scope of which is defined in the claims and their equivalents.

What is claimed is:

1. A shoe care apparatus comprising:
a cabinet;
a chamber disposed in the cabinet, the chamber being configured to accommodate shoes;
a machine room configured to accommodate a heat pump for heating air supplied to the chamber and dehumidifying air discharged from the chamber;
an air supply duct configured to connect the chamber and the machine room;
an air supply port formed on a sidewall of the chamber such that air flowing along the air supply duct is supplied into the chamber through the air supply port;
a support member disposed on the side wall of the chamber, the support member being disposed adjacent to the air supply port; and
a shoe hanger comprising a pair of support arms accommodated in the chamber, that are configured to be inserted into shoes, the shoe hanger being detachably mounted to the support member and having a hollow formed therein to allow air supplied from the air supply port to flow therethrough,
wherein the shoe hanger is provided with a sterilization device disposed inside the hollow, and
the support member is provided with a power supply part for applying power to the sterilization device.

2. The shoe care apparatus of claim 1, wherein the shoe hanger further comprises:
a shoe hanger body comprising the pair of support arms configured to protrude to support the shoes;
a passage formed inside the shoe hanger body while communicating with the air supply duct such that air moves to the pair of support arms; and
a plurality of holes connected to the passage and formed on outer surfaces of the pair of support arms configured to supply air into the chamber.

3. The shoe care apparatus of claim 2, wherein the sterilization device is disposed in the passage.

4. The shoe care apparatus of claim 2, wherein the sterilization device comprises at least one of an ultraviolet lamp or an ion generator.

5. The shoe care apparatus of claim 4, wherein the UV lamp or the ion generator is disposed inside the pair of support arms.

6. The shoe care apparatus of claim 5, wherein the pair of support arms comprises an installation bracket fixed to an inner upper surface of the pair of support arms such that the UV lamp or the ion generator is positioned in the passage.

7. The shoe care apparatus of claim 1, wherein the power supply part is configured to supply the sterilization device with power, and is disposed on at least one of an inner walls of a lateral side, an upper side, or a lower side of the support member.

8. The shoe care apparatus of claim 7, wherein the shoe hanger further comprises a power connection part that corresponds to the power supply part.

9. The shoe care apparatus of claim 8, wherein based on the shoe hanger being in a first position P1 of being coupled to the support member, the power connection part and the power supply part are in contact with each other, and the sterilization device is turned on.

10. The shoe care apparatus of claim 9, wherein based on the shoe hanger being in a second position P2 of being separated from the support member, the power connection part and the power supply part are separated from each other, and the sterilization device is turned off.

11. The shoe care apparatus of claim 9, wherein the power supply part comprises a contact terminal disposed on the at least one of the inner walls of the lateral side, the upper side, or the lower side of the support member, wherein the contact terminal is configured to be in contact with the power connection part.

12. The shoe care apparatus of claim 11, wherein the power supply part further comprises an elastic body configured to elastically support the contact terminal.

13. The shoe care apparatus of claim 1, further comprising a heat exchanger, configured to heat air introduced from the chamber, comprising a compressor ; and
a control part configured to alternatively turn on the compressor and the sterilization device.

14. The shoe care apparatus of claim 13, wherein the control part is configured to:
based on the compressor being turned off, turn on the sterilization device; and
based on the sterilization device being turned off, turn on the compressor.

15. The shoe care apparatus of claim 1, further comprising:
an output part comprising at least one of a display or a speaker; and
a control part configured to control the output part to output a visual notification or an audible notification notifying that the shoe hanger has been separated from the support member because power is not being applied to the sterilization device through the power supply part.
